SEQUENCE LISTING (74 sequences)

SEQ ID NO: 1 - protein, 33 aa, Artificial Sequence
  EGFR peptide
CDPSCPNGSCWGAGEENCQKLTKIICAQQCSGR

SEQ ID NO: 2 - protein, 7 aa, Artificial Sequence
  EGFR peptide
CEGPCRK

SEQ ID NO: 3 - protein, 7 aa, Artificial Sequence
  EGFR peptide
EDSFLQR

SEQ ID NO: 4 - protein, 10 aa, Artificial Sequence
  EGFR peptide
IPLENLQIIR

SEQ ID NO: 5 - protein, 13 aa, Artificial Sequence
  EGFR peptide
EISDGDVIISGNK

SEQ ID NO: 6 - protein, 15 aa, Artificial Sequence
  EGFR peptide
EITGFLLIQAWPENR

SEQ ID NO: 7 - protein, 12 aa, Artificial Sequence
  EGFR peptide
ELREATSPKANK

SEQ ID NO: 8 - protein, 8 aa, Artificial Sequence
  EGFR peptide
EYHAEGGK

SEQ ID NO: 9 - protein, 10 aa, Artificial Sequence
  EGFR peptide
FRELIIEFSK

SEQ ID NO: 10 - protein, 9 aa, Artificial Sequence
  EGFR peptide
GLWIPEGEK

SEQ ID NO: 11 - protein, 17 aa, Artificial Sequence
  EGFR peptide
GLWIPEGEKVKIPVAIK

SEQ ID NO: 12 - protein, 39 aa, Artificial Sequence
  EGFR peptide
HFKNCTSISGDLHILPVAFRGDSFTHTPPLDPQELDILK

SEQ ID NO: 13 - protein, 14 aa, Artificial Sequence
  EGFR peptide
IICAQQCSGRCRGK

SEQ ID NO: 14 - protein, 29 aa, Artificial Sequence
  EGFR peptide
IPSIATGMVGALLLLLVVALGIGLFMRRR

SEQ ID NO: 15 - protein, 6 aa, Artificial Sequence
  EGFR peptide
CEGPCR

SEQ ID NO: 16 - protein, 10 aa, Artificial Sequence
  EGFR peptide
LFGTSGQKTK

SEQ ID NO: 17 - protein, 13 aa, Artificial Sequence
  EGFR peptide
LTKIICAQQCSGR

SEQ ID NO: 18 - protein, 36 aa, Artificial Sequence
  EGFR peptide
NCTSISGDLHILPVAFRGDSFTHTPPLDPQELDILK

SEQ ID NO: 19 - protein, 19 aa, Artificial Sequence
  EGFR peptide
PYDGIPASEISSILEKGER

SEQ ID NO: 20 - protein, 21 aa, Artificial Sequence
  EGFR peptide
PAGSVQNPVYHNQPLNPAPSR

SEQ ID NO: 21 - protein, 26 aa, Artificial Sequence
  EGFR peptide
SPSDCCHNQCAAGCTGPRESDCLVCR

SEQ ID NO: 22 - protein, 8 aa, Artificial Sequence
  EGFR peptide
ITDFGLAK

SEQ ID NO: 23 - protein, 21 aa, Artificial Sequence
  EGFR peptide
TPQHVKITDFGLAKLLGAEEK

SEQ ID NO: 24 - protein, 11 aa, Artificial Sequence
  EGFR peptide
VCNGIGIGEFK

SEQ ID NO: 25 - protein, 25 aa, Artificial Sequence
  EGFR peptide
VCNGIGIGEFKDSLSINATNIKHFK

SEQ ID NO: 26 - protein, 23 aa, Artificial Sequence
  EGFR peptide
VLGSGAFGTVYKGLWIPEGEKVK

SEQ ID NO: 27 - protein, 13 aa, Artificial Sequence
  EGFR peptide
YSFGATCVKKCPR

SEQ ID NO: 28 - protein, 22 aa, Artificial Sequence
  EGFR peptide
CRGKSPSDCCHNQCAAGCTGPR

SEQ ID NO: 29 - protein, 24 aa, Artificial Sequence
  EGFR peptide
DIVSSDFLSNMSMDFQNHLGSCQK

SEQ ID NO: 30 - protein, 27 aa, Artificial Sequence
  EGFR peptide
EFVENSECIQCHPECLPQAMNITCTGR

SEQ ID NO: 31 - protein, 15 aa, Artificial Sequence
  EGFR peptide
ELIIEFSKMARDPQR

SEQ ID NO: 32 - protein, 19 aa, Artificial Sequence
  EGFR peptide
ELVEPLTPSGEAPNQALLR

SEQ ID NO: 33 - protein, 11 aa, Artificial Sequence
  EGFR peptide
ESDCLVCRKFR

SEQ ID NO: 34 - protein, 19 aa, Artificial Sequence
  EGFR peptide
GDSFTHTPPLDPQELDILK

SEQ ID NO: 35 - protein, 27 aa, Artificial Sequence
  EGFR peptide
GENSCKATGQVCHALCSPEGCWGPEPR

SEQ ID NO: 36 - protein, 28 aa, Artificial Sequence
  EGFR peptide
GKSPSDCCHNQCAAGCTGPRESDCLVCR

SEQ ID NO: 37 - protein, 16 aa, Artificial Sequence
  EGFR peptide
GRECVDKCNLLEGEPR

SEQ ID NO: 38 - protein, 9 aa, Artificial Sequence
  EGFR peptide
ILKETEFKK

SEQ ID NO: 39 - protein, 10 aa, Artificial Sequence
  EGFR peptide
IPLENLQIIR

SEQ ID NO: 40 - protein, 12 aa, Artificial Sequence
  EGFR peptide
KVCNGIGIGEFK

SEQ ID NO: 41 - protein, 23 aa, Artificial Sequence
  EGFR peptide
KVCNGIGIGEFKDSLSINATNIK

SEQ ID NO: 42 - protein, 24 aa, Artificial Sequence
  EGFR peptide
LLQERELVEPLTPSGEAPNQALLR

SEQ ID NO: 43 - protein, 13 aa, Artificial Sequence
  EGFR Peptide
MHLPSPTDSNFYR

SEQ ID NO: 44 - protein, 19 aa, Artificial Sequence
  EGFR Peptide
NVLVKTPQHVKITDFGLAK

SEQ ID NO: 45 - protein, 11 aa, Artificial Sequence
  EGFR Peptide
NVSRGRECVDK

SEQ ID NO: 46 - protein, 8 aa, Artificial Sequence
  EGFR Peptide
NYDLSFLK

SEQ ID NO: 47 - protein, 12 aa, Artificial Sequence
  EGFR Peptide
PKFRELIIEFSK

SEQ ID NO: 48 - protein, 24 aa, Artificial Sequence
  EGFR Peptide
LLQERELVEPLTPSGEAPNQALLR

SEQ ID NO: 49 - protein, 16 aa, Artificial Sequence
  EGFR Peptide
SLKEISDGDVIISGNK

SEQ ID NO: 50 - protein, 13 aa, Artificial Sequence
  EGFR Peptide
TDLHAFENLEIIR

SEQ ID NO: 51 - protein, 15 aa, Artificial Sequence
  EGFR Peptide
TDLHAFENLEIIRGR

SEQ ID NO: 52 - protein, 22 aa, Artificial Sequence
  EGFR Peptide
TKQHGQFSLAVVSLNITSLGLR

SEQ ID NO: 53 - protein, 9 aa, Artificial Sequence
  EGFR Peptide
TLRRLLQER

SEQ ID NO: 54 - protein, 21 aa, Artificial Sequence
  EGFR Peptide
TPLLSSLSATSNNSTVACIDR

SEQ ID NO: 55 - protein, 11 aa, Artificial Sequence
  EGFR Peptide
VAPQSSEFIGA

SEQ ID NO: 56 - protein, 9 aa, Artificial Sequence
  EGFR Peptide
YLVIQGDER

SEQ ID NO: 57 - protein, 11 aa, Artificial Sequence
  EGFR Peptide
GSTAENAEYLR

SEQ ID NO: 58 - protein, 11 aa, Artificial Sequence
  EGFR Peptide
  MOD_RES (9)..(9): PHOSPHORYLATION
GSTAENAEYLR

SEQ ID NO: 59 - protein, 20 aa, Artificial Sequence
  EGRF Peptide
GSHQISLDNPDYQQDDFFPK

SEQ ID NO: 60 - protein, 20 aa, Artificial Sequence
  EGRF Peptide
  MOD_RES (12)..(12): PHOSPHORYLATION
GSHQISLDNPDYQQDDFFPK

SEQ ID NO: 61 - protein, 21 aa, Artificial Sequence
  EGRF Peptide
PAGSVQNPVYHNQPLNPAPSR

SEQ ID NO: 62 - protein, 21 aa, Artificial Sequence
  EGRF Peptide
  MOD_RES (10)..(10): PHOSPHORYLATION
PAGSVQNPVYHNQPLNPAPSR

SEQ ID NO: 63 - protein, 19 aa, Artificial Sequence
  EGRF Peptide
ELVEPLTPSGEAPNQALLR

SEQ ID NO: 64 - protein, 19 aa, Artificial Sequence
  EGRF Peptide
  MOD_RES (9)..(9): PHOSPHORYLATION
ELVEPLTPSGEAPNQALLR

SEQ ID NO: 65 - protein, 19 aa, Artificial Sequence
  EGRF Peptide
  MOD_RES (7)..(7): PHOSPHORYLATION
ELVEPLTPSGEAPNQALLR

SEQ ID NO: 66 - protein, 19 aa, Artificial Sequence
  EGRF Peptide
  MOD_RES (7)..(7): PHOSPHORYLATION
  MOD_RES (9)..(9): PHOSPHORYLATION
ELVEPLTPSGEAPNQALLR

SEQ ID NO: 67 - protein, 19 aa, Artificial Sequence
  EGRF Peptide
GSHQISLDNPDYQQDFFPK

SEQ ID NO: 68 - protein, 19 aa, Artificial Sequence
  EGRF Peptide
  MOD_RES (12)..(12): PHOSPHORYLATION
GSHQISLDNPDYQQDFFPK

SEQ ID NO: 69 - protein, 30 aa, Artificial Sequence
  EGRF Peptide
YSDPTGALTEDSIDDTFLPVPEYINQSVPK

SEQ ID NO: 70 - protein, 30 aa, Artificial Sequence; identical to SEQ ID NO: 69
  EGRF Peptide
  MOD_RES (23)..(23): PHOSPHORYLATION

SEQ ID NO: 71 - protein, 30 aa, Artificial Sequence; identical to SEQ ID NO: 69
  EGRF Peptide
  MOD_RES (1)..(1): PHOSPHORYLATION

SEQ ID NO: 72 - protein, 30 aa, Artificial Sequence; identical to SEQ ID NO: 69
  EGRF Peptide
  MOD_RES (1)..(1): PHOSPHORYLATION
  MOD_RES (23)..(23): PHOSPHORYLATION

SEQ ID NO: 73 - protein, 8 aa, Artificial Sequence
  EGRF Peptide
EYHAEGGK

SEQ ID NO: 74 - protein, 8 aa, Artificial Sequence
  EGRF Peptide
  MOD_RES (2)..(2): PHOSPHORYLATION
EYHAEGGK

(19) United States
(12) Patent Application Publication
(10) Pub. No.: US 2017/0248608 A1
(43) Pub. Date: Aug. 31, 2017

(54) Epidermal Growth Factor Receptor (EGFR) Protein SRM Assay

(71) Applicant: EXPRESSION PATHOLOGY, INC., Rockville, MD (US)

(72) Inventors: David B. KRIZMAN, Gaithersburg, MD (US); Todd HEMBROUGH, Gaithersburg, MD (US); Sheeno THYPARAMBIL, Frederick, MD (US)

(21) Appl. No.: 15/444,310

(22) Filed: Feb. 27, 2017

Related U.S. Application Data
(63) Continuation of application No. 13/529,897, filed on Jun. 21, 2012, now abandoned.
Application No. 13/529,897 is a continuation of application No. PCT/US2010/061916, filed on Dec. 22, 2010.
(60) Provisional application No. 61/289,384, filed on Dec. 22, 2009.

(51) Int. Cl. G01N 33/68; A61K 31/517; A61K 31/5377; G01N 33/574; C07K 16/28

(52) U.S. Cl. CPC G01N 33/6863; G01N 33/6848; G01N 33/57407; C07K 16/2863; A61K 31/5377; A61K 31/517; G01N 2333/485; G01N 2560/00; C07K 2317/24

(57) ABSTRACT

The current disclosure provides for specific peptides, and derived ionization characteristics of the peptides, from the Epidermal Growth Factor Receptor (EGFR) protein that are particularly advantageous for quantifying the EGFR protein directly in biological samples that have been fixed in formalin by the method of Selected Reaction Monitoring (SRM) mass spectrometry, or what can also be termed as Multiple Reaction Monitoring (MRM) mass spectrometry. Such biological samples are chemically preserved and fixed wherein said biological sample is selected from tissues and cells treated with formaldehyde containing agents/fixatives including formalin-fixed tissue/cells, formalin-fixed/paraffin embedded (FFPE) tissue/cells, FFPE tissue blocks and cells from those blocks, and tissue culture cells that have been formalin fixed and or paraffin embedded. A protein sample is prepared from said biological sample using the Liquid Tissue™ reagents and protocol and the EGFR protein is quantitated in the Liquid Tissue™ sample by the method of SRM/MRM mass spectrometry by quantitating in the protein sample at least one or more of the peptides described. These peptides can be quantitated if they reside in a modified or an unmodified form. An example of a modified form of an EGFR peptide is phosphorylation of a tyrosine, threonine, serine, and/or other amino acid residues within the peptide sequence.

This application claims the benefit of U.S. Provisional Application 61/289,384, filed Dec. 22, 2009, entitled “Epidermal Growth Factor Receptor (EGFR) Protein SRM/MRM Assay” naming as an inventor David B. Krizman, the entirety of which is incorporated by reference.

### INTRODUCTION

Specific peptides derived from subsequences of the Epidermal Growth Factor Receptor protein and which will be referred to as EGFR, and which can also be referred to as the receptor tyrosine-protein kinase ErbB-1 protein, are provided. The peptide sequence and fragmentation/transition ions for each peptide are particularly useful in a mass spectrometry-based Selected Reaction Monitoring (SRM), which can also be referred to as a Multiple Reaction Monitoring (MRM) assay, and will be referred to as SRM/MRM. The use of one such peptide for SRM/MRM quantitative analysis of the EGFR protein is described.

This SRM/MRM assay can be used to measure relative or absolute quantitative levels of one or more of the specific peptides from the EGFR protein and therefore provide a means of measuring the amount of the EGFR protein in a given protein preparation obtained from a biological sample by mass spectrometry.

More specifically, the SRM/MRM assay can measure these peptides directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by references in their entirety. The methods described in U.S. Pat. No. 7,473,532 may conveniently be carried out using Liquid Tissue™ reagents and protocol available from Expression Pathology Inc. (Rockville, Md.).

The most widely and advantageously available form of tissues from cancer patients tissue is formalin fixed, paraffin embedded tissue. Formaldehyde/formalin fixation of surgically removed tissue is by far and away the most common method of preserving cancer tissue samples worldwide and is the accepted convention for standard pathology practice. Aqueous solutions of formaldehyde are referred to as formalin. “100%” formalin consists of a saturated solution of formaldehyde (this is about 40% by volume or 37% by mass) in water, with a small amount of stabilizer, usually methanol to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods to analyze formalin fixed cancer tissue will be the most accepted and heavily utilized methods for analysis of cancer patient tissue.

Results from the SRM/MRM assay can be used to correlate accurate and precise quantitative levels of the EGFR protein within the specific tissue samples (e.g., cancer tissue sample) of the patient or subject from whom the tissue (biological sample) was collected and preserved. This not only provides diagnostic information about the cancer, but also permits a physician or other medical professional to determine appropriate therapy for the patient. Such an assay that provides diagnostically and therapeutically important information about levels of protein expression in a diseased tissue or other patient sample is termed a companion diagnostic assay. For example, such an assay can be designed to diagnose the stage or degree of a cancer and determine a therapeutic agent to which a patient is most likely to respond.

### SUMMARY

The assays described herein measure relative or absolute levels of specific unmodified peptides from the EGFR protein and also can measure absolute or relative levels of specific modified peptides from the EGFR protein. Examples of modifications include phosphorylated amino acid residues and glycosylated amino acid residues that are present on the peptides.

Relative quantitative levels of the EGFR protein are determined by the SRM/MRM methodology for example by comparing SRM/MRM signature peak areas (e.g., signature peak area or integrated fragment ion intensity) of an individual EGFR peptide in different samples. Alternatively, it is possible to compare multiple SRM/MRM signature peak areas for multiple EGFR signature peptides, where each peptide has its own specific SRM/MRM signature peak, to determine the relative EGFR protein content in one biological sample with the EGFR protein content in one or more additional or different biological samples. In this way, the amount of a particular peptide, or peptides, from the EGFR protein, and therefore the amount of the EGFR protein, is determined relative to the same EGFR peptide, or peptides, across 2 or more biological samples under the same experimental conditions. In addition, relative quantitation can be determined for a given peptide, or peptides, from the EGFR protein within a single sample by comparing the signature peak area for that peptide by SRM/MRM methodology to the signature peak area for another and different peptide, or peptides, from a different protein, or proteins, within the same protein preparation from the biological sample. In this way, the amount of a particular peptide from the EGFR protein, and therefore the amount of the EGFR protein, is determined relative one to another within the same sample. These approaches generate quantitation of an individual peptide, or peptides, from the EGFR protein to the amount of another peptide, or peptides, between samples and within samples wherein the amounts as determined by signature peak area are relative one to another, regardless of the absolute weight to volume or weight to weight amounts of the EGFR peptide in the protein preparation from the biological sample. Relative quantitative data about individual signature peak areas between different samples are normalized to the amount of protein analyzed per sample. Relative quantitation can be performed across many peptides from multiple proteins and the EGFR protein simultaneously in a single sample and/or across many samples to gain insight into relative protein amounts, one peptide/protein with respect to other peptides/proteins.

Absolute quantitative levels of the EGFR protein are determined by, for example, the SRM/MRM methodology whereby the SRM/MRM signature peak area of an individual peptide from the EGFR protein in one biological sample is compared to the SRM/MRM signature peak area of a spiked internal standard. In one embodiment, the internal standard is a synthetic version of the same exact EGFR peptide that contains one or more amino acid residues labeled with one or more heavy isotopes. Such isotope labeled internal standards are synthesized so that when analyzed by mass spectrometry it generates a predictable and consistent SRM/MRM signature peak that is different and distinct from the native EGFR peptide signature peak and which can be used as a comparator peak. Thus when the internal standard is spiked into a protein preparation from a biological sample in known amounts and analyzed by mass spectrometry, the SRM/MRM signature peak area of the native peptide is compared to the SRM/MRM signature peak area of the internal standard peptide, and this numerical comparison indicates either the absolute molarity and/or absolute weight of the native peptide present in the original protein preparation from the biological sample. Absolute quantitative data for fragment peptides are displayed according to the amount of protein analyzed per sample. Absolute quantitation can be performed across many peptides, and thus proteins, simultaneously in a single sample and/or across many samples to gain insight into absolute protein amounts in individual biological samples and in entire cohorts of individual samples.

The SRM/MRM assay method can be used to aid diagnosis of the stage of cancer, for example, directly in patient-derived tissue, such as formalin fixed tissue, and to aid in determining which therapeutic agent would be most advantageous for use in treating that patient. Cancer tissue that is removed from a patient either through surgery, such as for therapeutic removal of partial or entire tumors, or through biopsy procedures conducted to determine the presence or absence of suspected disease, is analyzed to determine whether or not a specific protein, or proteins, and which forms of proteins, are present in that patient tissue. Moreover, the expression level of a protein, or multiple proteins, can be determined and compared to a “normal” or reference level found in healthy tissue. Normal or reference levels of proteins found in healthy tissue may be derived from, for example, the relevant tissues of one or more individuals that do not have cancer. Alternatively, normal or reference levels may be obtained for individuals with cancer by analysis of relevant tissues not affected by the cancer. Assays of protein levels (e.g., EGFR levels) can also be used to diagnose the stage of cancer in a patient or subject diagnosed with cancer by employing the EGFR levels. Levels or amounts of proteins or peptides can be defined as the quantity expressed in moles, mass or weight of a protein or peptide determined by the SRM/MRM assay. The level or amount may be normalized to total the level or amount of protein or another component in the lysate analyzed (e.g., expressed in micromoles/microgram of protein or micrograms/microgram of protein). In addition, the level or amount of a protein or peptide may be determined on volume basis, expressed, for example, in micromolar or nanograms/microliter. The level or amount of protein or peptide as determined by the SRM/MRM assay can also be normalized to the number of cells analyzed. Information regarding EGFR can thus be used to aid in determining stage or grade of a cancer by correlating the level of the EGFR protein (or fragment peptides of the EGFR protein) with levels observed in normal tissues. Once the stage and/or grade, and/or EGFR protein expression characteristics of the cancer has been determined, that information can be matched to a list of therapeutic agents (chemical and biological) developed to specifically treat cancer tissue that is characterized by, for example, abnormal expression of the protein or protein(s) (e.g., EGFR) that were assayed. Matching information from an EGFR protein assay to a list of therapeutic agents that specifically targets, for example, the EGFR protein or cells/tissue expressing the protein, defines what has been termed a personalized medicine approach to treating disease. The assay methods described herein form the foundation of a personalized medicine approach by using analysis of proteins from the patient's own tissue as a source for diagnostic and treatment decisions.

### BRIEF DESCRIPTION OF THE DRAWINGS

FIG. 1, parts A to C, show an example of SRM/MRM assay of a single peptide from the EGFR protein performed on Liquid Tissue™ lysates with quantitation of the EGFR peptide conducted on a triplequadrupole mass spectrometer.

### DETAILED DESCRIPTION

In principle, any predicted peptide derived from EGFR protein, prepared for example by digesting with a protease of known specificity (e.g. trypsin), can be used as a surrogate reporter to determine the abundance of EGFR protein in a sample using a mass spectrometry-based SRM/MRM assay. Similarly, any predicted peptide sequence containing an amino acid residue at a site that is known to be potentially modified in EGFR protein also might potentially be used to assay the extent of modification of EGFR protein in a sample.

EGFR fragment peptides may be generated by a variety of means including by the use of the Liquid Tissue™ protocol provided in U.S. Pat. No. 7,473,532. The Liquid Tissue™ protocol and reagents are capable of producing peptide samples suitable for mass spectroscopic analysis from formalin fixed paraffin embedded tissue by proteolytic digestion of the proteins in the tissue/biological sample. In the Liquid Tissue™ protocol the tissue/biological is heated in a buffer for an extended period of time (e.g., from about 80° C. to about 100° C. for a period of time from about 10 minutes to about 4 hours) to reverse or release protein cross-linking. The buffer employed is a neutral buffer, (e.g., a Tris-based buffer, or a buffer containing a detergent). Following heat treatment the tissue/biological sample is treated with one or more proteases, including but not limited to trypsin, chymotrypsin, pepsin, and endoproteinase Lys-C for a time sufficient to disrupt the tissue and cellular structure of said biological sample and to liquefy said sample (e.g., a period of time from 30 minutes to 24 hours at a temperature from 37° C. to 65° C.). The result of the heating and proteolysis is a liquid, soluble, dilutable biomolecule lysate.

Surprisingly, it was found that many potential peptide sequences from the EGFR protein are unsuitable or ineffective for use in mass spectrometry-based SRM/MRM assays for reasons that are not immediately evident. As it was not possible to predict the most suitable peptides for MRM/SRM assay, it was necessary to experimentally identify modified and unmodified peptides in actual Liquid Tissue™ lysates to develop a reliable and accurate SRM/MRM assay for the EGFR protein. While not wishing to be bound by any theory, it is believed that some peptides might, for example, be difficult to detect by mass spectrometry as they do not ionize well or produce fragments distinct from other proteins, peptides may also fail to resolve well in separation (e.g., liquid chromatography), or adhere to glass or plastic ware.

EGFR peptides found in various embodiments of this disclosure (e.g., Tables 1 and 2) were derived from the EGFR protein by protease digestion of all the proteins within a complex Liquid Tissue™ lysate prepared from cells procured from formalin fixed cancer tissue. Unless noted otherwise, in each instance the protease was trypsin. The Liquid Tissue™ lysate was then analyzed by mass spectrometry to determine those peptides derived from the EGFR protein that are detected and analyzed by mass spectrometry. Identification of a specific preferred subset of peptides for mass-spectrometric analysis is based on; 1) experimental determination of which peptide or peptides from a protein ionize in mass spectrometry analyses of Liquid Tissue™ lysates, and 2) the ability of the peptide to survive the protocol and experimental conditions used in preparing a Liquid Tissue™ lysate. This latter property extends not only to the amino acid sequence of the peptide but also to the ability of a modified amino acid residue within a peptide to survive in modified form during the sample preparation.

TABLE 1

| Table 1 | |
|---|---|
| SEQ ID | Peptide Sequence |
| SEQ ID NO: 1 | CDPSCPNGSCWGAGEENCQKLTKIICAQQCSGR |
| SEQ ID NO: 2 | CEGPCRK |
| SEQ ID NO: 3 | EDSFLQR |
| SEQ ID NO: 4 | IPLENLQIIR |
| SEQ ID NO: 5 | EISDGDVIISGNK |
| SEQ ID NO: 6 | EITGFLLIQAWPENR |
| SEQ ID NO: 7 | ELREATSPKANK |
| SEQ ID NO: 8 | EYHAEGGK |
| SEQ ID NO: 9 | FRELIIEFSK |
| SEQ ID NO: 10 | GLWIPEGEK |
| SEQ ID NO: 11 | GLWIPEGEKVKIPVAIK |
| SEQ ID NO: 12 | HFKNCTSISGDLHILPVAFRGDSFTHTPPLDP |
| | QELDILK |
| SEQ ID NO: 13 | IICAQQCSGRCRGK |
| SEQ ID NO: 14 | IPSIATGMVGALLLLLVVALGIGLFMRRR |
| SEQ ID NO: 15 | CEGPCR |
| SEQ ID NO: 16 | LFGTSGQKTK |
| SEQ ID NO: 17 | LTKIICAQQCSGR |
| SEQ ID NO: 18 | NCTSISGDLHILPVAFRGDSFTHTPPLDPQEL |
| | DILK |
| SEQ ID NO: 19 | PYDGIPASEISSILEKGER |
| SEQ ID NO: 20 | PAGSVQNPVYHNQPLNPAPSR |
| SEQ ID NO: 21 | SPSDCCHNQCAAGCTGPRESDCLVCR |
| SEQ ID NO: 22 | ITDFGLAK |
| SEQ ED NO: 23 | TPQHVKITDFGLAKLLGAEEK |
| SEQ ID NO: 24 | VCNGIGIGEFK |
| SEQ ID NO: 25 | VCNGIGIGEFKDSLSINATNIKHFK |
| SEQ ID NO: 26 | VLGSGAFGTVYKGLWIPEGEKVK |
| SEQ ID NO: 27 | YSFGATCVKKCPR |
| SEQ ID NO: 28 | CRGKSPSDCCHNQCAAGCTGPR |
| SEQ ID NO: 29 | DIVSSDFLSNMSMDFQNHLGSCQK |
| SEQ ID NO: 30 | EFVENSECIQCHPECLPQAMNITCTGR |
| SEQ ID NO: 31 | ELIIEFSKMARDPQR |
| SEQ ID NO: 32 | ELVEPLTPSGEAPNQALLR |
| SEQ ID NO: 33 | ESDCLVCRKFR |
| SEQ ID NO: 34 | GDSFTHTPPLDPQELDILK |
| SEQ ID NO: 35 | GENSCKATGQVCHALCSPEGCWGPEPR |
| SEQ ID NO: 36 | GKSPSDCCHNQCAAGCTGPRESDCLVCR |
| SEQ ID NO: 37 | GRECVDKCNLLEGEPR |
| SEQ ID NO: 38 | ILKETEFKK |
| SEQ ID NO: 39 | IPLENLQIIR |
| SEQ ID NO: 40 | KVCNGIGIGEFK |
| SEQ ID NO: 41 | KVCNGIGIGEFKDSLSINATNIK |
| SEQ ID NO: 42 | LLQERELVEPLTPSGEAPNQALLR |
| SEQ ID NO: 43 | MHLPSPTDSNFYR |
| SEQ ID NO: 44 | NVLVKTPQHVKITDFGLAK |
| SEQ ID NO: 45 | NVSRGRECVDK |
| SEQ ID NO: 46 | NYDLSFLK |
| SEQ ID NO: 47 | PKFRELIIEFSK |
| SEQ ID NO: 48 | LLQERELVEPLTPSGEAPNQALLR |
| SEQ ID NO: 49 | SLKEISDGDVIISGNK |
| SEQ ID NO: 50 | TDLHAFENLEIIR |
| SEQ ID NO: 51 | TDLHAFENLEIIRGR |
| SEQ ID NO: 52 | TKQHGQFSLAVVSLNITSLGLR |
| SEQ ID NO: 53 | TLRRLLQER |
| SEQ ID NO: 54 | TPLLSSLSATSNNSTVACIDR |
| SEQ ID NO: 55 | VAPQSSEFIGA- |
| SEQ ID NO: 56 | YLVIQGDER |
| SEQ ID NO: 57 | GSTAENAEYLR |
| SEQ ID NO: 58 | GSTAENAEY[Phosphoryl]LR |
| SEQ ID NO: 59 | GSHQISLDNPDYQQDDFFPK |
| SEQ ID NO: 60 | GSHQISLDNPDY[Phosphoryl]QQDDFFPK |
| SEQ ID NO: 61 | PAGSVQNPVYHNQPLNPAPSR |
| SEQ ID NO: 62 | PAGSVQNPVY[Phosphoryl]HNQPLNPAPSR |
| SEQ ID NO: 63 | ELVEPLTPSGEAPNQALLR |
| SEQ ID NO: 64 | ELVEPLTPS[Phosphoryl]GEAPNQALLR |
| SEQ ID NO: 65 | ELVEPLT[Phosphoryl]PSGEAPNQALLR |
| SEQ ID NO: 66 | ELVEPLT[Phosphoryl]PS[Phosphoryl] |
| | GEAPNQALLR |
| SEQ ID NO: 67 | GSHQISLDNPDYQQDFFPK |
| SEQ ID NO: 68 | GSHQISLDNPDY[Phosphoryl]QQDFFPK |
| SEQ ID NO: 69 | YSDPTGALTEDSIDDTFLPVPEYINQSVPK |
| SEQ ID NO: 70 | YSDPTGALTEDSIDDTFLPVPEY[Phosphoryl] |
| | INQSVPK |
| SEQ ID NO: 71 | Y[Phosphoryl]SDPTGALTEDSIDDTFLPVPEY |
| | INQSVPK |
| SEQ ID NO: 72 | Y[Phosphoryl]SDPTGALTEDSIDDTFLPVPEY |
| | [Phosphoryl]INQSVPK |
| SEQ ID NO: 73 | EYHAEGGK |
| SEQ ID NO: 74 | EY[Phosphoryl]HAEGGK |

| Table 2 | | Mono | Precursor | | | |
|---|---|---|---|---|---|---|
| SEQ ID | | Isotopic | Charge | Precursor | Transition | |
| NO | Peptide sequence | Mass | State | m/z | m/z | Ion Type |
| SEQ ID | GLWIPEGEK | 1027.534 | 2 | 514.774 | 559.272 | y5 |
| NO: 10 | | | 2 | 514.774 | 672.356 | y6 |
| | | | 2 | 514.774 | 858.435 | y7 |
| SEQ ID | GSTAENAEYLR | 1209.562 | 2 | 605.7880249 | 651.346 | y5 |
| NO: 57 | | | 2 | 605.7880249 | 765.388 | y6 |
| | | | 2 | 605.7880249 | 894.431 | y7 |
| SEQ ID | ITDFGLAK | 863.475 | 2 | 432.744 | 535.323 | y5 |
| NO: 22 | | | 2 | 432.744 | 650.350 | y6 |
| | | | 2 | 432.744 | 751.398 | y7 |
| SEQ ID | IPLENLQIIR | 1207.737 | 2 | 604.872 | 756.472 | y6 |
| NO: 39 | | | 2 | 604.872 | 885.515 | y7 |
| | | | 2 | 604.872 | 998.599 | y8 |
| SEQ ID | GSTAENAEY[Phosphoryl]LR | 1289.529 | 2 | 645.7709961 | 596.7833 | Reporter |
| NO: 58 | | | 2 | 645.7709961 | 660.2747 | y4 |
| | | | 2 | 645.7709961 | 731.3118 | y5 |
| | | | 2 | 645.7709961 | 845.3547 | y6 |
| | | | 2 | 645.7709961 | 974.3973 | y7 |
| | | | 2 | 645.7709961 | 1045.434 | y8 |
| | | | 2 | 645.7709961 | 1146.482 | y9 |
| | | | 2 | 645.7709961 | 1233.514 | y10 |
| | | | 2 | 645.7709961 | 1290.536 | y11 |
| SEQ ID | ELVEPLTPS[Phosphoryl]GE | 2113.046 | 3 | 705.3549805 | 406.2426 | y7 |
| NO: 64 | APNQALLR | | 3 | 705.3549805 | 811.4779 | y7 |
| | | | 3 | 705.3549805 | 822.411 | y15 |
| SEQ ID | ELVEPLT[Phosphoryl]PS | 2113.046 | 2 | 1057.530029 | 1008.542 | Reporter |
| NO: 65 | GEAPNQALLR | | 2 | 1057.530029 | 1252.664 | y12 |
| SEQ ID | GSHQISLDNPD | 2314.99 | 2 | 1158.501953 | 1364.555 | y10 |
| NO: 68 | Y[Phosphoryl]QQDFFPK | | 2 | 1158.501953 | 1478.598 | y11 |

Protein lysates from cells procured directly from formalin (formaldehyde) fixed tissue were prepared using the Liquid Tissue™ reagents and protocol that entails collecting cells into a sample tube via tissue microdissection followed by heating the cells in the Liquid Tissue™ buffer for an extended period of time. Once the formalin-induced cross linking has been negatively affected, the tissue/cells are then digested to completion in a predictable manner using a protease, as for example including but not limited to the protease trypsin. Each protein lysate is turned into a collection of peptides by digestion of intact polypeptides with the protease. Each Liquid Tissue™ lysate was analyzed (e.g., by ion trap mass spectrometry) to perform multiple global proteomic surveys of the peptides where the data was presented as identification of as many peptides as could be identified by mass spectrometry from all cellular proteins present in each protein lysate. An ion trap mass spectrometer or another form of a mass spectrometer that is capable of performing global profiling for identification of as many peptides as possible from a single complex protein/peptide lysate is employed. Ion trap mass spectrometers however may be the best type of mass spectrometer for conducting global profiling of peptides. Although SRM/MRM assay can be developed and performed on any type of mass spectrometer, including a MALDI, ion trap, or triple quadrupole, the most advantageous instrument platform for SRM/MRM assay is often considered to be a triple quadrupole instrument platform.

Once as many peptides as possible were identified in a single MS analysis of a single lysate under the conditions employed, then that list of peptides was collated and used to determine the proteins that were detected in that lysate. That process was repeated for multiple Liquid Tissue™ lysates, and the very large list of peptides was collated into a single dataset. That type of dataset can be considered to represent the peptides that can be detected in the type of biological sample that was analyzed (after protease digestion), and specifically in a Liquid Tissue™ lysate of the biological sample, and thus includes the peptides for specific proteins, such as for example the EGFR protein.

In one embodiment, the EGFR tryptic peptides identified as useful in the determination of absolute or relative amounts of the EGFR receptor include one or more, two or more, three or more, four or more, five or more, six or more, eight or more, or ten or more of the peptides of SEQ ID NO:1, SEQ ID NO:2, SEQ ID NO:3, SEQ 1D NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:38, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, and SEQ ID NO:74, each of which are listed in Table 1. Each of those peptides was detected by mass spectrometry in Liquid Tissue™ lysates prepared from formalin fixed, paraffin embedded tissue. Thus, each of the peptides in Table 1, or any combination of those peptides (e.g., one or more, two or more, three or more, four or more, five or more, six or more, eight or more, or ten or more of those peptides recited in Table 1, and particularly combinations with one or more of the peptides found in table 2) are candidates for use in quantitative SRM/MRM assay for the EGRF protein in human biological samples, including directly in formalin fixed patient tissue.

The EGFR tryptic peptides listed in Table 1 include those detected from multiple Liquid Tissue™ lysates of multiple different formalin fixed tissues of different human organs including prostate, colon, and breast. Each of those peptides is considered useful for quantitative SRM/MRM assay of the EGFR protein in formalin fixed tissue. Further data analysis of these experiments indicated no preference is observed for any specific peptides from any specific organ site. Thus, each of these peptides is believed to be suitable for conducting SRM/MRM assays of the EGFR protein on a Liquid Tissue™ lysate from any formalin fixed tissue originating from any biological sample or from any organ site in the body.

In one embodiment the peptides in Table 1, or any combination of those peptides (e.g., one or more, two or more, three or more, four or more, five or more, six or more, eight or more, or nine or more of those peptides recited in Table 1, and particularly combinations with the peptides also found in Table 2) are assayed by methods that do not rely upon mass spectroscopy, including, but not limited to, immunological methods (e.g., Western blotting or ELISA). Regardless of how information directed to the amount of the peptide(s) (absolute or relative) is obtained, the information may be employed in any of the methods described herein, including indicating (diagnosing) the presence of cancer in a subject, determining the stage/grade/status of the cancer, providing a prognosis, or determining the therapeutics or treatment regimen for a subject/patient.

Embodiments of the present disclosure includes compositions comprising one or more, two or more, three or more, four or more, five or more, six or more, eight or more, or ten or more of the peptides in Table 1. In some embodiments, the compositions comprise one or more, two or more, three or more, four or more, five or more, six or more, or seven or more of the peptides in Table 2. Compositions comprising peptides may include one or more, two or more, three or more, four or more, five or more, six or more, eight or more, or ten or more peptides that are isotopically labeled. Each of the peptides may be labeled with one or more isotopes selected independently from the group consisting of: ¹⁸O, ¹⁷O, ³⁴S, ¹⁵N, ¹³C, ²H or combinations thereof. Compositions comprising peptides from the EGFR protein, whether isotope labeled or not, do not need to contain all of the peptides from that protein (e.g., a complete set of tryptic peptides). In some embodiments the compositions do not contain one or more, two or more, three or more, four or more, five or more, six or more, eight or more, or ten or more peptides from EGFR, and particularly peptides appearing in Table 1 or Table 2. Compositions comprising peptides may be in the form of dried or lyophized materials, liquid (e.g., aqueous) solutions or suspensions, arrays, or blots.

An important consideration for conducting an SRM/MRM assay is the type of instrument that may be employed in the analysis of the peptides. Although SRM/MRM assays can be developed and performed on any type of mass spectrometer, including a MALDI, ion trap, or triple quadrupole, the most advantageous instrument platform for SRM/MRM assay is often considered to be a triple quadrupole instrument platform. That type of a mass spectrometer may be considered to be the most suitable instrument for analyzing a single isolated target peptide within a very complex protein lysate that may consist of hundreds of thousands to millions of individual peptides from all the proteins contained within a cell.

In order to most efficiently implement SRM/MRM assay for each peptide derived from the EGFR protein it is desirable to utilize information in addition to the peptide sequence in the analysis. That additional information may be used in directing and instructing the mass spectrometer (e.g. a triple quadrupole mass spectrometer), to perform the correct and focused analysis of specific targeted peptide(s), such that the assay may be effectively performed.

The additional information about target peptides in general, and about specific EGFR peptides, may include one or more of the mono isotopic mass of the peptide, its precursor charge state, the precursor m/z value, the m/z transition ions, and the ion type of each transition ion. Additional peptide information that may be used to develop an SRM/MRM assay for the EGFR protein is shown by example for eight (8) of the EGFR peptides from the list in Table 1 and is shown in Table 2. Similar additional information described for the eight (8) EGFR peptides shown by example in Table 2 may be prepared, obtained, and applied to the analysis of the other peptides contained in Table 1.

The method described below was used to: 1) identify candidate peptides from the EGFR protein that can be used for a mass spectrometry-based SRM/MRM assay for the EGFR protein, 2) develop individual SRM/MRM assay, or assays, for target peptides from the EGFR protein in order to correlate and 3) apply quantitative assays to cancer diagnosis and/or choice of optimal therapy.

### Assay Method

1. Identification of SRM/MRM candidate fragment peptides for the EGFR protein
   a. Prepare a Liquid Tissue™ protein lysate from a formalin fixed biological sample using a protease or proteases, (that may or may not include trypsin), to digest proteins
   b. Analyze all protein fragments in the Liquid Tissue™ lysate on an ion trap tandem mass spectrometer and identify all fragment peptides from the EGFR protein, where individual fragment peptides do not contain any peptide modifications such as phosphorylations or glycosylations
   c. Analyze all protein fragments in the Liquid Tissue™ lysate on an ion trap tandem mass spectrometer and identify all fragment peptides from the EGFR protein that carry peptide modifications such as for example phosphorylated or glycosylated residues
   d. All peptides generated by a specific digestion method from the entire, full length EGFR protein potentially can be measured, but preferred peptides used for development of the SRM/MRM assay are those that are identified by mass spectrometry directly in a complex Liquid Tissue™ protein lysate prepared from a formalin fixed biological sample
   e. Peptides that are specifically modified (phosphorylated, glycosylated, etc.) in patient tissue and which ionize, and thus detected, in a mass spectrometer when analyzing a Liquid Tissue™ lysate from a formalin fixed biological sample are identified as candidate peptides for assaying peptide modifications of the EGFR protein
2. Mass Spectrometry Assay for Fragment Peptides from EGFR Protein
   a. SRM/MRM assay on a triple quadrupole mass spectrometer for individual fragment peptides identified in a Liquid Tissue™ lysate is applied to peptides from the EGFR protein
      i. Determine optimal retention time for a fragment peptide for optimal chromatography conditions including but not limited to gel electrophoresis, liquid chromatography, capillary electrophoresis, nano-reversed phase liquid chromatography, high performance liquid chromatography, or reverse phase high performance liquid chromatography
      ii. Determine the mono isotopic mass of the peptide, the precursor charge state for each peptide, the precursor m/z value for each peptide, the m/z transition ions for each peptide, and the ion type of each transition ion for each fragment peptide in order to develop an SRM/MRM assay for each peptide.
      iii. SRM/MRM assay can then be conducted using the information from (i) and (ii) on a triple quadrupole mass spectrometer where each peptide has a characteristic and unique SRM/MRM signature peak that precisely defines the unique SRM/MRM assay as performed on a triple quadrupole mass spectrometer
   b. Perform SRM/MRM analysis so that the amount of the fragment peptide of the EGFR protein that is detected, as a function of the unique SRM/MRM signature peak area from an SRM/MRM mass spectrometry analysis, can indicate both the relative and absolute amount of the protein in a particular protein lysate.
      i. Relative quantitation may be achieved by:
         1. Determining increased or decreased presence of the EGFR protein by comparing the SRM/MRM signature peak area from a given EGFR peptide detected in a Liquid Tissue™ lysate from one formalin fixed biological sample to the same SRM/MRM signature peak area of the same EGFR fragment peptide in at least a second, third, fourth or more Liquid Tissue™ lysates from least a second, third, fourth or more formalin fixed biological samples
         2. Determining increased or decreased presence of the EGFR protein by comparing the SRM/MRM signature peak area from a given EGFR peptide detected in a Liquid Tissue™ lysate from one formalin fixed biological sample to SRM/MRM signature peak areas developed from fragment peptides from other proteins, in other samples derived from different and separate biological sources, where the SRM/MRM signature peak area comparison between the 2 samples for a peptide fragment are normalized to amount of protein analyzed in each sample.
         3. Determining increased or decreased presence of the EGFR protein by comparing the SRM/MRM signature peak area for a given EGFR peptide to the SRM/MRM signature peak areas from other fragment peptides derived from different proteins within the same Liquid Tissue™ lysate from the formalin fixed biological sample in order to normalize changing levels of EGFR protein to levels of other proteins that do not change their levels of expression under various cellular conditions.
         4. These assays can be applied to both unmodified fragment peptides and for modified fragment peptides of the EGFR protein, where the modifications include but are not limited to phosphorylation and/or glycosylation, and where the relative levels of modified peptides are determined in the same manner as determining relative amounts of unmodified peptides.
      ii. Absolute quantitation of a given peptide may be achieved by comparing the SRM/MRM signature peak area for a given fragment peptide from the EGFR protein in an individual biological sample to the SRM/MRM signature peak area of an internal fragment peptide standard spiked into the protein lysate from the biological sample
         1. The internal standard is a labeled synthetic version of the fragment peptide from the EGFR protein that is being interrogated. This standard is spiked into a sample in known amounts, and the SRM/MRM signature peak area can be determined for both the internal fragment peptide standard and the native fragment peptide in the biological sample separately, followed by comparison of both peak areas
         2. This can be applied to unmodified fragment peptides and modified fragment peptides, where the modifications include but are not limited to phosphorylation and/or glycosylation, and where the absolute levels of modified peptides can be determined in the same manner as determining absolute levels of unmodified peptides,
3. Apply Fragment Peptide Quantitation to Cancer Diagnosis and Treatment
   a. Perform relative and/or absolute quantitation of fragment peptide levels of the EGFR protein and demonstrate that the previously-determined association, as well understood in the field of cancer, of EGFR protein expression to the stage/grade/status of cancer in patient tumor tissue is confirmed
   b. Perform relative and/or absolute quantitation of fragment peptide levels of the EGFR protein and demonstrate correlation with clinical outcomes from different treatment strategies, wherein this correlation has already been demonstrated in the field or can be demonstrated in the future through correlation studies across cohorts of patients and tissue from those patients. Once either previously established correlations or correlations derived in the future are confirmed by this assay then the assay method can be used to determine optimal treatment strategy

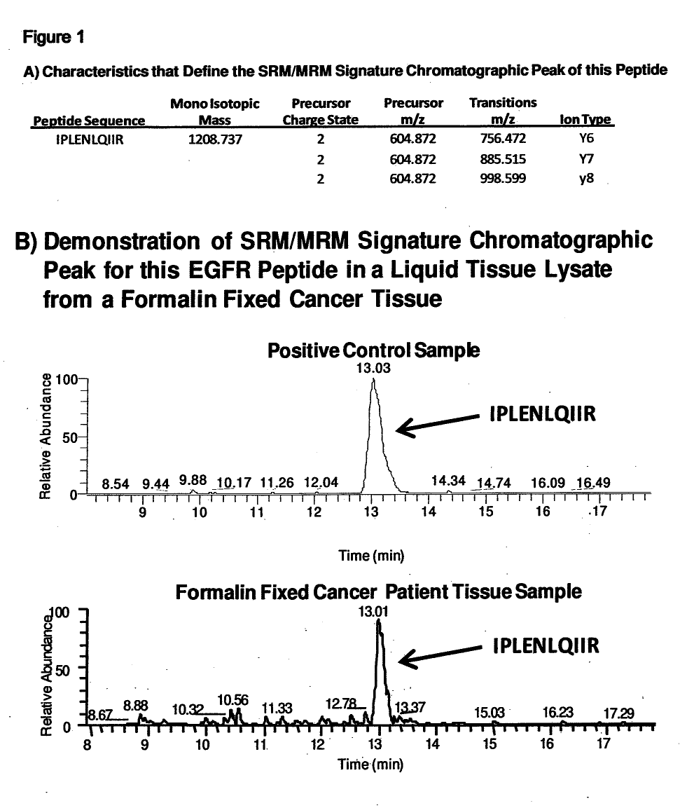

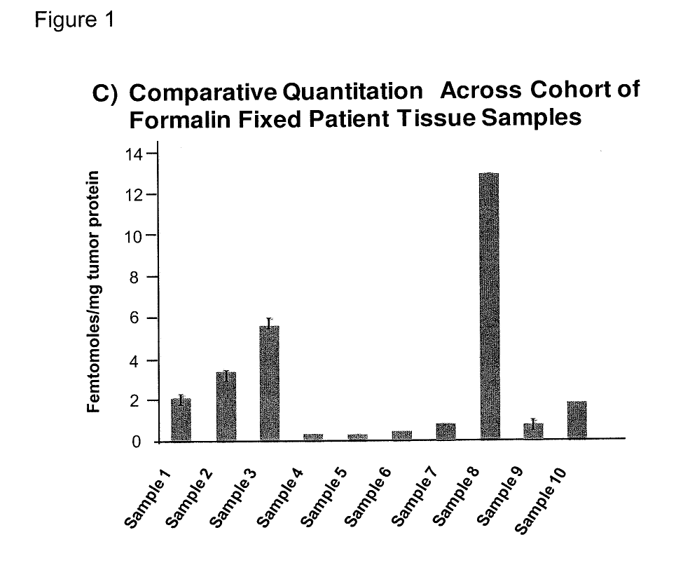

FIG. 1 shows an example of a single SRM/MRM assay performed on Liquid Tissue™ lysates from formalin fixed cancer tissue. An SRM/MRM assay was developed for a single peptide for quantitation of the EGFR protein on a triple quadrupole mass spectrometer. Specific and unique characteristics about this EGFR peptide (sequence IPLENLQIIR) were developed by analysis of all EGFR peptides on both an ion trap and triple quadrupole mass spectrometers and are shown in FIG. 1A. That information includes the monoisotopic mass of the peptide, its precursor charge state, the precursor m/z value, the transition m/z values of the precursor, and the ion types of each of the identified transitions. That information must be determined experimentally for each and every candidate SRM/MRM peptide directly in Liquid Tissue™ lysates from formalin fixed tissue; because, interestingly, not all peptides from the EGFR protein can be detected in such lysates using SRM/MRM as described herein, indicating that EGFR peptides not detected cannot be considered candidate peptides for developing an SRM/MRM assay for use in quantitating peptides/proteins directly in Liquid Tissue™ lysates from formalin fixed tissue.

As shown in FIG. 1B, this particular SRM/MRM assay was performed on a triple quadrupole mass spectrometer. A control protein lysate where the peptide was known to be present in large amounts was analyzed because this lysate was prepared from a mouse xenograft tumor that resulted from injection of a human-derived cancer cell line into a nude mouse. Thus this xenograft tumor was the positive control. The experimental sample in this experiment was a Liquid Tissue™ protein lysate prepared from standard formalin fixed, paraffin embedded human breast cancer tissue. Data from the assay indicates the presence of the unique SRM/MRM signature peak for this EGFR peptide in both the control sample and the experimental sample. By comparing the SRM/MRM signature peak area between these 2 samples generates relative quantitative measure for the EGFR protein between 2 different biological samples.

FIG. 1C shows quantitative measurement of the above-mentioned peptide across a collection of ten (10) formalin fixed cancer tissues using an internal standard to achieve absolute quantitation of the EGFR protein across a cohort of cancer-derived patient samples. These data indicate absolute amounts of this EGFR peptide as a function of molar amount of the peptide per microgram of protein lysate analyzed. Assessment of EGFR protein levels in tissues based on analysis of formalin fixed patient-derived tissue can provide diagnostic, prognostic, and therapeutically-relevant information about each particular patient. In one embodiment, this disclosure describes a method for measuring the level of the Epidermal Growth Factor Receptor (EGFR) protein in a biological sample, comprising detecting and/or quantifying the amount of one or more modified or unmodified EGFR fragment peptides in a protein digest prepared from said biological sample using mass spectrometry; and calculating the level of modified or unmodified EGFR protein in said sample; and wherein said level is a relative level or an absolute level. In a related embodiment, quantifying one or more EGFR fragment peptides comprises determining the amount of the each of the EGFR fragment peptides in a biological sample by comparison to an added internal standard peptide of known amount, wherein each of the EGFR fragment peptides in the biological sample is compared to an internal standard peptide having the same amino acid sequence. In some embodiments the internal standard is an isotopically labeled internal standard peptide comprises one or more heavy stable isotopes selected from ¹⁸O, ¹⁷O, ³⁴s, ¹⁵N, ¹³C, ²H or combinations thereof.

The method for measuring the level of the EGFR protein in a biological sample described herein (or fragment peptides as surrogates thereof) may be used as a diagnostic indicator of cancer in a patient or subject. In one embodiment, the results from measurements of the level of the EGFR protein may be employed to determine the diagnostic stage/grade/status of a cancer by correlating (e.g., comparing) the level of EGFR receptor found in a tissue with the level of that protein found in normal and/or cancerous or precancerous tissues.

1. A method for measuring the level of the Epidermal Growth Factor Receptor (EGFR) protein in a biological sample of formalin fixed tissue sample from a human subject, comprising detecting and quantifying by mass spectrometry the amount of an EGFR fragment peptide in a protein digest prepared from said biological sample using mass spectrometry; and calculating the level of modified or unmodified EGFR protein in said sample; wherein said EGFR fragment peptide consists of the amino acid sequence SEQ ID NO:4 and
wherein said level is a relative level or an absolute level.

2. The method of claim 1, further comprising the step of fractionating said protein digest prior to detecting and/or quantifying the amount of one or more modified or unmodified EGFR fragment peptides.

3. The method of claim 2, wherein said fractionating step is selected from the group consisting of gel electrophoresis, liquid chromatography, capillary electrophoresis, nano-reversed phase liquid chromatography, high performance liquid chromatography, or reverse phase high performance liquid chromatography.

4. (canceled)

5. The method of claim 1, wherein said protein digest comprises a protease digest.

6. The method of claim 5, wherein said protein digest comprises a trypsin digest.

7-11. (canceled)

12. The method of claim 1, wherein the tissue is paraffin embedded tissue.

13. The method of claim 1, wherein the tissue is obtained from a tumor.

14-16. (canceled)

17. The method of claim 1, wherein quantifying the EGFR fragment peptide comprises comparing an amount of said EGFR fragment peptide to the amount of the same EGFR fragment peptide in a different and separate biological sample.

18. The method of claim 1, wherein quantifying said EGFR fragment peptide comprises determining the amount of said EGFR fragment peptide in a biological sample by comparison to an added internal standard peptide of known amount, wherein said EGFR fragment peptide in the biological sample is compared to an internal standard peptide having the same amino acid sequence.

19. The method of claim 18, wherein the internal standard peptide is an isotopically labeled peptide.

20. The method of claim 19, wherein the isotopically labeled internal standard peptide comprises one or more heavy stable isotopes selected from ¹⁸O, ¹⁷O, ³⁴S, ¹⁵N, ¹³C, ²H or combinations thereof.

21. The method of claim 1, wherein detecting and quantifying the amount of said EGFR fragment peptide in the protein digest indicates the presence of EGFR protein and an association with cancer in the subject.

22. The method of claim 21, further comprising correlating the results of said detecting and quantifying the amount of said EGFR fragment peptide, or the level of said EGFR protein to the diagnostic stage/grade/status of the cancer.

23. The method of claim 22, wherein correlating the results of said detecting and quantifying the amount of said EGFR fragment peptide, or the level of said EGFR protein to the diagnostic stage/grade/status of the cancer is combined with detecting and/or quantifying the amount of other proteins or peptides from other proteins in a multiplex format to provide additional information about the diagnostic stage/grade/status of the cancer.

24. The method of claim 1, further comprising selecting for the subject from which said biological sample was obtained a treatment based on the presence, absence, or amount of said EGFR fragment peptide or the level of EGFR protein.

25. The method of claim 24, further comprising administering to the patient from which said biological sample was obtained a therapeutically effective amount of a therapeutic agent, wherein the therapeutic agent and/or amount of the therapeutic agent administered is based upon the amount of said EGFR fragment peptide or the level of EGFR protein.

26. The method of claim 26, wherein said therapeutic agent binds the EGFR protein and/or inhibits its biological activity,

27. The method of claim 26, wherein the therapeutic agent is selected from Tarceva, Iressa, and Erbitux.

28-32. (canceled)
